SEQUENCE LISTING (2 sequences)

SEQ ID NO: 1 - DNA, 1439 bp, synthetic construct
  source 1..1439: mol_type = other DNA
ctagctcctaaaaggttaccccaccggctttgggtgttacaaactctcatggtgcgatgggcggtgtgtacaaggcccgggaacgtattcaccgcggcatgctgatccgcgattactagcgattccgacttcgtgtaggcgagttgcagcctacagtccgaactgagaatggttttaagagattagcttaacctcgcggtctcgcgactcgttgtaccatccattgtagcacgtgtgtagcccaggtcataaggggcatgatgatttgacgtcgtccccaccttcctccggtttgtcaccggcagtctcactagagtgcccaacttaatgctggcaactagtaataagggttgcgctcgttgcgggacttaacccaacatctcacgacacgagctgacgacaaccatgcaccacctgtcattctgtccccgaagggaacctctaatctcttagactgtcagaagatgtcaagacctggtaaggttcttcgcgtagcttcgaattaaaccacatgctccaccgcttgtgcgggcccccgtcaattcttttgagtttcaaccttgcggtcgtactccccaggcggattacttaatgcgttagctgcagcactgaagggcggaaaccctccaacacttagtaatcatcgtttacggcatggactaccagggtatctaatcctgttcgctacccatgctttcgagcctcagcgtcagttgcagaccagacagccgccttcgccactggtgttcttccatatatctacgcatttcaccgctacacatggagttccactgtcctcttctgcactcaagtctcccagtttccaatgcacttcttcggttgagccgaaggctttcacattagacttaaaagaccgcctgcgctcgctttacgcccaataaatccggataacgcttgccacctacgtattaccgcggctgctggcacgtagttagccgtggctttctggttaaataccgtcactgggtaaacagttactcttacccacgttcttctttaacaacagagctttacgagccgaaacccttcttcactcacgcggcgttgctccatcagacttgcgtccattgtggaagattccctactgctgcctcccgtaggagtctgggccgtgtctcagtcccaatgtggccgattaccctctcaggtcggctacgtatcactgccttggtgagcctttacctcaccaactagctaatacgccgcgggtccatccagaagtgatagcagagccatcttttaaaagaaaaccatgcggttttctctgttatacggtattagcatctgtttccaggtgttatcccctacttctgggcaggttacccacgtgttactcacccgttcgccactcacttcgtgttaaagtctcaatcagtacaagtacgtcataatcaattaacggaagttcgttcgactgc

SEQ ID NO: 2 - DNA, 1489 bp, synthetic construct
  source 1..1489: mol_type = other DNA
gacgaacgctggcggcgtgcctaatacatgcaagttgagcgctgaaggttggtacttgtaccgactggatgagcagcgaacgggtgagtaacgcgtggggaatctgcctttgagcgggggacaacatttggaaacgaatgctaataccgcataaaaactttaaacacaagttttaagtttgaaagatgcaattgcatcactcaaagatgatcccgcgttgtattagctagttggtgaggtaaaggctcaccaaggcgatgatacatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttggtagagaagaacgttggtgagagtggaaagctcatcaagtgacggtaactacccagaaagggacggctaactacgtgccagcagccgcggtaatacgtaggtcccgagcgttgtccggatttattgggcgtaaagcgagcgcaggtggtttattaagtctggtgtaaaaggcagtggctcaaccattgtatgcattggaaactggtagacttgagtgcaggagaggagagtggaattccatgtgtagcggtgaaatgcgtagatatatggaggaacaccggtggcgaaagcggctctctggcctgtaactgacactgaggctcgaaagcgtggggagcaaacaggattagataccctggtagtccacgccgtaaacgatgagtgctagatgtagggagctataagttctctgtatcgcagctaacgcaataagcactccgcctggggagtacgaccgcaaggttgaaactcaaaggaattgacgggggcccgcacaagcggtggagcatgtggtttaattcgaagcaacgcgaagaaccttaccaggtcttgacatactcgtgctattcctagagataggaagttccttcgggacacgggatacaggtggtgcatggttgtcgtcagctcgtgtcgtgagatgttgggttaagtcccgcaacgagcgcaacccctattgttagttgccatcattaagttgggcactctaacgagactgccggtgataaaccggaggaaggtggggatgacgtcaaatcatcatgccccttatgacctgggctacacacgtgctacaatggatggtacaacgagtcgcgagacagtgatgtttagctaatctcttaaaaccattctcagttcggattgtaggctgcaactcgcctacatgaagtcggaatcgctagtaatcgcggatcagcacgccgcggtgaatacgttcccgggccttgtacacaccgcccgtcacaccacgggagttgggagtacccgaagtaggttgcctaaccgcaaggagggcgcttcctaaggtaagaccgatgactggggtgaagtcgtaacaaggtagccg

(19) United States
(12) Patent Application Publication
(10) Pub. No.: US 2023/0380454 A1
(43) Pub. Date: Nov. 30, 2023

(54) Endophytic Pediococcus pentosaceus EL5 in alfalfa and application thereof

(71) Applicant: CHINA AGRICULTURAL UNIVERSITY, Beijing (CN)

(72) Inventors: Kuikui NI, Beijing (CN); Hongzhang ZHOU, Beijing (CN); Fuyu YANG, Beijing (CN)

(73) Assignee: CHINA AGRICULTURAL UNIVERSITY, Beijing (CN)

(21) Appl. No.: 18/325,900

(22) Filed: May 30, 2023

(30) Foreign Application Priority Data
May 30, 2022 (CN) 202210614489.6

(51) Int. Cl. A23K 30/18; C12N 1/20; A23K 10/12

(52) U.S. Cl. CPC A23K 30/18; C12N 1/205; A23K 10/12; C12R 2001/01

(57) ABSTRACT

The present application relates to a Pediococcus pentosaceus and application thereof. The Pediococcus pentosaceus is Pediococcus pentosaceus EL5 with deposit number of CGMCC No. 23923. The Pediococcus pentosaceus EL5 of an embodiment of the present application can be used for silage to improve the activity of lactic acid bacteria and the quality of silage fermentation.

### CROSS-REFERENCE TO RELATED APPLICATIONS

This application claims priority to Chinese Patent Application No. 202210614489.6, filed on May 30, 2022, which is hereby incorporated by reference in their entirety.

### REFERENCE TO ELECTRONIC SEQUENCE LISTING

This application contains a sequence listing which has been submitted electronically in .XML format and is hereby incorporated by reference in its entirety. Said .XML copy, created on May 29, 2023, is named “Endophytic_Pediococcus_pentosaceus EL5_in_alfalfa_and_application_thereof.xml” and is 5,768 bytes in size, which is a copy of the sequence listing as filed in Chinese Patent Application No. 202210614489.6. The sequence listing contained in this XML file is part of the specification and is hereby incorporated by reference herein in its entirety.

### TECHNICAL FIELD

The present application relates to microbial feed, and in particular to an endophytic Pediococcus pentosaceus EL5 in alfalfa suitable for preparing silage, especially alfalfa silage.

### BACKGROUND ART

Alfalfa is a perennial herbaceous plant belonging to the leguminous alfalfa genus, which is drought resistant and mainly distributed in the northwest, north China, northeast, and Changjiang-Huaihe River Valley of China. It has a cultivation history of over 2000 years in China. Alfalfa has the advantages of high protein content and good palatability, making it a high-quality forage source for ruminants, especially cows. The demand for alfalfa forage in China's animal husbandry industry is increasing, and it heavily relies on imports from abroad. The contradiction between supply and demand is becoming increasingly prominent, which seriously restricts the development of China's animal husbandry industry.

Silage, as a simple and low-cost storage method for forage, can effectively preserve the nutritional of forage, reduce nutrient loss, and improve palatability and digestibility of forage. It has become an important component of ruminant diets worldwide. Making silage from alfalfa can not only keep the green characteristics of alfalfa grass, but also reduce its nutrient loss, which is one of the effective measures to solve the shortage of forage in winter and spring. The key to silage lies in the degree of lactic acid fermentation dominated by lactic acid bacteria. In order for lactic acid bacteria to grow and reproduce effectively, it is necessary to provide suitable growth environments and conditions. Microorganisms and their living environment mutually select and co evolve. Currently, conventional exogenous lactic acid bacteria additives have low activity during alfalfa fermentation due to poor adaptability to raw material, which is an important factor restricting alfalfa silage.

### SUMMARY OF THE INVENTION

In a first aspect, an embodiment of the present application provides a Pediococcus pentosaceus EL5 with the deposit number of CGMCC No. 23923.

According to an embodiment of the present application, the Pediococcus pentosaceus EL5 comprises 16S rRNA as shown in SEQ ID NO. 1.

According to an embodiment of the present application, the Pediococcus pentosaceus EL5 is isolated from the phyllosphere endophytic of alfalfa.

In a second aspect, an embodiment of the present application provides a silage additive, which comprises the aforementioned Pediococcus pentosaceus EL5.

In a third aspect, an embodiment of the present application provides a silage, which comprises the aforementioned Pediococcus pentosaceus EL5.

According to an embodiment of the present application, the silage is alfalfa silage.

In a fourth aspect, an embodiment of the present application provides a preparation method of the aforementioned silage, which comprises: mixing silage raw material with the aforementioned Pediococcus pentosaceus EL5 and fermenting to produce the silage.

According to an embodiment of the present application, the ratio of the number of viable strains of Pediococcus pentosaceus EL5 to the mass of the silage raw material is 1.0×10⁵˜2.0×10⁶ CFU/g.

According to an embodiment of the present application, the silage raw material includes alfalfa.

Fifth, an embodiment of the present application provides an application of the aforementioned Pediococcus pentosaceus EL5 in the preparation of silage.

Compared with the prior art, the present application can achieve at least one of the following beneficial effects:
1. The Pediococcus pentosaceus EL5 in the embodiment of the present application can be used for silage to improve the activity of lactic acid bacteria and the quality of silage fermentation.
2. The silage of an embodiment of the present application improves the fermentation quality of silage through Pediococcus pentosaceus EL5, which has the advantages of low cost, safe, reliable and easy to use.

In the present application, the above technical schemes can also be combined with each other to realize more preferred combination schemes. Other features and advantages of the present application will be described in the following instructions, and some advantages may become apparent from the instructions or be understood by implementing the present application. The object and other advantages of the present application can be realized and obtained through the contents specially pointed out in the instructions and the accompanying drawings.

### BRIEF DESCRIPTION OF DRAWINGS

The drawings are only to show specific embodiments and are not considered as a limitation of the present application. Wherein:

FIG. 1 shows the acid production and growth rate diagram of endophytic Pediococcus pentosaceus EL5 in alfalfa in an embodiment;

FIG. 2 shows the acid production and growth rate diagram of epiphytic Lactobacillus lactis L2 in alfalfa in a comparative example;

FIG. 3 shows a comparison diagram of carbon source utilization rates between the endophytic Pediococcus pentosaceus EL5 in alfalfa in the embodiment and the epiphytic Lactobacillus lactis L2 in alfalfa in the comparative example.

### DETAILED DESCRIPTION OF THE INVENTION

The preferred embodiments of the present application are described in detail below in combination with the accompanying drawings. The accompanying drawings form part of the application and, together with the embodiments of the present application, are used to explain the principle of the present application, not to limit the scope of the present application.

An embodiment of the present application provides an endophytic Pediococcus pentosaceus EL5 in alfalfa, whose strain is deposited in the China General Microbiological Culture Collection Center (CGMCC, address: Institute of Microbiology Chinese Academy of Sciences, No. 3, Yard 1, West Beichen Road, Chaoyang District, Beijing), with the deposit number of CGMCC No. 23923, and the date of deposit is Nov. 16, 2021.

In one embodiment, the 16S rRNA of the Pediococcus pentosaceus EL5 is shown in SEQ ID NO. 1.

The Pediococcus pentosaceus EL5 in an embodiment of the present application is isolated from phyllosphere endophytic of alfalfa, wherein the alfalfa tissue is subjected to strict surface disinfection before extraction and separation.

In one embodiment, the isolation and cultivation process of the Pediococcus pentosaceus EL5 includes:

After sterilization treatment, the alfalfa is subjected to compression and/or crushing treatment (such as grinding) to allow internal juice to flow out;

Mix the treated alfalfa with 0.85% salt water, then diluted and coated the supernatant to MRS agar medium for cultivation.

In an embodiment, the growth temperature of the EL5 strain can be 15-45° C., and further can be 30-35° C., such as 20° C., 25° C., 27° C., 33° C., 37° C., and 40° C.

In an embodiment, the EL5 strain can reduce the pH value of the MRS medium to 3.5-4.0 after 24 hours of cultivation.

The biological characteristics of the endophytic Pediococcus pentosaceus EL5 in alfalfa in an embodiment of the present application are Gram-positive cocci, dextrose homomorphic fermentation, and can growth normally under pH=4.0, indicating strong acid resistance. It can grow well in environments with 3% and 6.5% salt concentrations, indicating strong salt tolerance. After 24 hours of cultivation at 30° C. in MRS liquid medium, the OD600 is 1.5524, indicating a fast growth rate. By testing the utilization rates of 95 carbon sources in Biolog AN microporous plates at 30° C., the total carbon sources utilization rate of EL5 in 48 hours is 103.22% higher than that of epiphytic Lactobacillus lactis L2 in alfalfa, indicating a high total utilization rate of different carbon sources.

An embodiment of the application provides a silage additive, which includes the aforementioned Pediococcus pentosaceus EL5, wherein the Pediococcus pentosaceus EL5 is the active component of the additive.

In an embodiment, the silage additive can be the additive of alfalfa silage.

An embodiment of the present application provides a silage, which includes the aforementioned Pediococcus pentosaceus EL5.

In an embodiment, the silage can be alfalfa silage.

An embodiment of the present application provides a method for preparing the silage, including: mixing silage raw material with the aforementioned Pediococcus pentosaceus EL5 and fermenting to produce the silage.

In an embodiment, the silage raw material can be alfalfa.

In the silage of an embodiment, the ratio of the number of viable bacteria of Pediococcus pentosaceus EL5 to the mass of the silage raw material is 1.0×10⁵ CFU/g˜2.0×10⁶ CFU/g, e.g. 2.0×10⁵ CFU/g, 5.0×10⁵ CFU/g, 8.0×10⁵ CFU/g, 1.0×10⁶ CFU/g, or 1.5×10⁶ CFU/g.

The preparation method of the silage according to an embodiment of the present application includes the following steps:
S1: Cut alfalfa raw material and mix evenly;
S2: Add the Pediococcus pentosaceus EL5 to the alfalfa raw material, and the number of effective viable strains of Pediococcus pentosaceus EL5 added to per gram of alfalfa raw material is more than 1.0×10⁶.
S3: Store the alfalfa system with the addition of Pediococcus pentosaceus EL5 under vacuum sealing.

In an embodiment, in step S1, the manually cut alfalfa raw material can be chopped to 2-3 cm.

In step S3 of an embodiment, the alfalfa system can be stored at a temperature of 20-25° C., and the pH value of the system can be reduced to 4.69-4.78 after 60 days of storage, such as 4.70, 4.75.

An embodiment of the present application further provides an application of the aforementioned Pediococcus pentosaceus EL5 in preparation of the silage.

In an embodiment of the present application, Pediococcus pentosaceus EL5 is acid-resistant, has a fast grow rate, and has the highest utilization rate of carbon source; furthermore, it has strong comprehensive acid production ability, and can quickly generate lactic acid, reduce the content of pH and ammonia nitrogen, improve the palatability of silage, and inhibit the growth of Escherichia coli and fungi, and is conducive to improving its aerobic stability and extending the storage life of silage. The nutritional quality of the alfalfa silage raw material is retained to the greatest extent, and the effect of the prepared silage is better than that of the alfalfa epiphytic strain, and the biological safety is good with low preparation cost.

The Pediococcus pentosaceus EL5 according to an embodiment of the present application, can overcome the problem of adaptability of lactic acid bacteria to alfalfa raw material during silage, thereby improving the activity of lactic acid bacteria and improving the quality of silage fermentation.

The Pediococcus pentosaceus EL5 according to an embodiment of the present application has excellent performance in normal silage environments and is more effective than the epiphytic lactic acid bacteria strains derived from plants.

The silage or its preparation method according to an embodiment of the present application adopts plant endophytic lactic acid bacteria, which can overcome the problem of adaptability of lactic acid bacteria to alfalfa raw material during silage compared with other exogenous lactic acid bacteria, improve the activity of lactic acid bacteria and the quality of silage fermentation.

The silage or its preparation method according to an embodiment of the present application uses lactic acid bacteria to improve the fermentation quality of silage, which has the advantages of low cost, safe, reliable and easy to use.

In the following, the Pediococcus pentosaceus EL5 and silage of an embodiment of the present application are further described in combination with the drawings and specific embodiments. Wherein, the alfalfa material used was alfalfa collected from Fengmao Shengyuan Agricultural Technology Co., Ltd. in Huanghua City, Hebei Province. The MRS liquid mediums used in the isolation and culture, and determination of growth rate, acid production rate and other physiological and biochemical indicators of endophytic Pediococcus pentosaceus EL5 in alfalfa included: 10.0 g of Proteose peptone NO. 3, 10.0 g of beef extract, 5.0 g of yeast extract, 20.0 g of dextrose, 1 mL of polysorbate 80, 2.0 g of ammonium citrate, 5.0 g of sodium acetate (NaAc), 0.1 g of magnesium sulfate (MgSO₄·7H₂O), 0.05 g of manganese sulfate (MnSO₄·4H₂O), 2.0 g of potassium hydrogen phosphate (K₂HPO₄), and 1000 mL of distilled water (H₂O). On the basis of MRS liquid culture medium, add 15 g of agar to form MRS solid culture medium. All the above culture mediums were sterilized in a high-pressure sterilization pot at 121° C. for 20 minutes.

### Embodiment

Isolation and Culture of Endophytic Pediococcus pentosaceus EL5 in Alfalfa

Put fresh alfalfa material into sterile sampling bags and bring it back to the laboratory. Weigh 10 g of the entire alfalfa plant in a sterile environment on a super clean workbench and perform surface disinfection operations: immerse it in 70% ethanol for 90 seconds, 3.25-4% sodium hypochlorite solution for 120 seconds, and 70% ethanol for 30 seconds in turn. Rinse it with sterile water three times and use sterile filter paper to absorb surface water, and coat the sterile water from the last rinse as a control.

Put the sterilized material into a sterilized mortar for grinding (a little sterilized quartz sand was added to facilitate grinding, and calcium carbonate was used as a buffer), grind the alfalfa tissue, add 9 mL of 8.5% sterile physiological saline, fully stir them, and then stand for 3 min, take 1 mL of supernatant (denoted as 10⁻¹ concentration gradient) and dilute it to 10⁻² concentration gradient and 10⁻³ concentration gradient in turn with 10 times of dilution degree. Take 100 uL of each gradient diluent and apply it to MRS solid culture medium for plate coating. Place the plate in an anaerobic bag and incubate it in a 30° C. or 37° C. incubator.

After the growth of colonies, select a single colony and repeatedly perform plate marking separation until a single colony EL5 is obtained. Inoculate the EL5 with an inoculation needle onto the inclined surface of the MRS solid culture medium test tube and store it in a refrigerator at 4° C.

Morphological Identification of Endophytic Pediococcus pentosaceus EL5 in Alfalfa

The testing procedures of Gram staining, cell shape observation, and catalase experiment are as follows:

Gram staining method: Take a ring of water in the center of a slide, then use an inoculation ring to pick a small amount of bacteria and mix them evenly with water droplets on the slide, and apply them into a thin bacterial film for natural drying. Fix the glass slide upwards on a low flame: after 1 minute of initial staining with crystal violet, rinse thoroughly with water (with gentle movements to avoid direct impact of water flow on the bacterial block). Drip iodine solution for 1-2 minutes for mordant dyeing, decolorize with 95% ethanol and wash with water, and redye with saffron for 1-2 minutes then washing with water; natural drying after that; Observe with 1000× oil microscope: blue purple represents Gram positive bacteria, while red represents Gram negative bacteria.

Catalase experiment: Use a gun to suck 3% (volume fraction) hydrogen peroxide solution onto a plate, use an inoculation ring to pick up a small amount of bacteria, and mix it thoroughly with the hydrogen peroxide solution. After 2-3 minutes, if bubbles are observed, it is positive, and if no bubbles are observed, it is negative.

Physiological and Biochemical Testing of Endophytic Pediococcus pentosaceus EL5 in Alfalfa

According to experiments such as growth temperature (4, 15, 30, 35, 45° C.) and growth pH (3, 3.5, 4.0, 4.5, 9.0), the endophytic Pediococcus pentosaceus EL5 in alfalfa was tested. The procedures were as follows:

Determination and screening of acid production and growth rate of lactic acid bacteria: the isolated and purified strains were inoculated into 3 mL of MRS liquid medium, and incubated overnight at 30° C. and 250 rpm on a shaking table for about 14-16 hours. Transfer 1% (V/V) of the inoculation amount into a new 3 mL MRS liquid culture medium, and culture it in the shaking table at 30° C. and 250 rpm. Measure the pH value of the MRS liquid culture medium at 0, 2, 4, 6, 8, 10, 12, 14, 16, 18, 20, 22, and 24 hours after the inoculation time respectively, and measure the absorbance value at a wavelength of 600 nm. Each strain of bacteria requires 3 replicates at each time point, and the test tubes for cultivating the bacteria need to be of the same specification.

Temperature tolerance test: Transfer 1% (V/V) of the inoculation amount into a new MRS liquid culture medium and incubate at 4° C., 15° C., 30° C., 35° C., and 45° C. respectively for 2 days.

pH tolerance test: Inoculate 1% (V/V) of the inoculum to MRS liquid culture mediums with pH 3.0, 3.5, 4.0, 4.5, and 9.0 respectively at 30° C. for 2 days (regulated with 2.0M NaOH or 1.0M HCL).

Salt tolerance: Lactic acid bacteria were inoculated into MRS liquid culture mediums with NaCl contents of 3% and 6.5%, respectively, and then placed in a 30° C. incubator for 2 days to observe the growth status of the lactic acid bacteria.

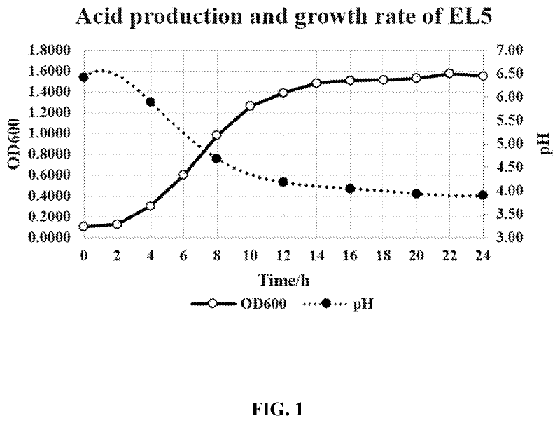

The morphology and physiological and biochemical characteristics of strain EL5 are shown in Table 1 and FIG. 1.

TABLE 1
The morphology and physiological and biochemical
characteristics of strain EL5

| | |
|---|---|
| Strain | EL5 |
| Source | Endophytic bacteria of alfalfa |
| Shape | Sphere |
| Gram staining | + |
| Catalase | − |
| Fermentation type | Homotype |

Growth temperature and corresponding results

| | |
|---|---|
| 4° C. | w |
| 15° C. | ++ |
| 30° C. | +++ |
| 35° C. | +++ |
| 45° C. | + |

Growth pH value and corresponding results

| | |
|---|---|
| 3.0 | w |
| 3.5 | w |
| 4.0 | + |
| 4.5 | ++ |
| 9.0 | w |

Salt resistance and corresponding results

| | |
|---|---|
| 3% NaCl | ++ |
| 6.5% NaCl | + |

In the results of Table 1, the “w”, “+”, “++”, and “+++” in the tolerance test represent the following meanings: w: OD600ₙₘ<0.5; +: 0.5≤OD600ₙₘ<1; ++: 1≤OD600ₙₘ<1.5; +++: 1.5≤OD600ₙₘ.

The results in FIG. 1 and Table 1 indicate that the strain of Pediococcus pentosaceus EL5 is a gram-positive, homofermentative coccus with strong acid and salt tolerance, fast growth rate, and strong comprehensive acid-producing ability.

Homology Analysis of 16S rRNA Gene of Endophytic Pediococcus pentosaceus EL5 in Alfalfa

Incubate the strain in 5 mL MRS medium overnight at 35° C., then transfer the bacterial solution into a 1.5 mL centrifuge tube, centrifuge at 10000 rpm/min for 3-5 minutes to collect the bacteria. Wash twice with TE 0.1 (10 mmol/L Tris HCL, 0.1 mmol/L EDTA, pH 8.0), and then extract DNA using the TIANAmp Bacteria DNA Kit (TIANGEN BIOTECH CO., LTD, Beijing, China) kit. The absorbance value was measured at OD600ₙₘ.

Afterwards, PCR amplification was performed, with primers of 27f and 1492r for 16S rRNA (Monis et al., 2005). The PCR reaction was at 95° C. (5 minutes) −94° C. (30 seconds) −55° C. (1 minute) −72° C. (1.5 minutes) −72° C. (10 minutes), with a reaction cycle of 30 times at 94° C. (30 seconds) −55° C. (1 minute) −72° C. (1.5 minutes). The amplified products were sent to Meiji Biotechnology Co., Ltd. (China) for sequencing, and the results were compared in the NCBI gene bank to identify standard strains such as Pedosaccus pentosaceus that are closely related to this strain. DNAman software was used to analyze the similarity between the selected strain's partial sequence (about 1400 bp-1500 bp) of 16S rRNA (see SEQ ID NO. 1) and the standard strain, and the similarity between EL5 and Pedosaccus pentosaceus exceeded 99%. Combined with physiological and biochemical indicators, it is determined that EL5 is of the same species as Pedosaccus pentosaceus.

### Comparative Example

Isolation and Culture of Epiphytic Lactococcus lactis L2 in Alfalfa

Put fresh alfalfa material into sterile sampling bags and bring it back to the laboratory. Weigh 20 g of the entire alfalfa plant in 180 mL of sterile physiological saline in a sterile environment on a super clean workbench, shake at 180 r/min and room temperature on a shaking table for 2 hours, and obtain a stock solution with a concentration of 100 g·L⁻¹, denoted as 10⁻¹ gradient. Then, take 1 mL of this stock solution and add it to a test tube containing 9 mL of sterile distilled water, and shake evenly to make the final concentration of 10 g·L⁻¹, denoted as a 10⁻² gradient. Use this method to sequentially dilute to 10⁻⁴ gradient, respectively. Take 100 uL of each gradient dilution solution for plate coating on MRS solid culture mediums, and place them in a 30° C. or 37° C. incubator for cultivation. Wherein, the MRS medium formulation is the same as the embodiment. After the growth of colonies, select a single colony and repeatedly perform plate marking separation until a single colony L2 is obtained. Inoculate the single colony with an inoculation needle onto the inclined surface of the MRS solid culture medium test tube and store it in a refrigerator at 4° C.

Identification and testing of Epiphytic Lactococcus lactis L2 in Alfalfa

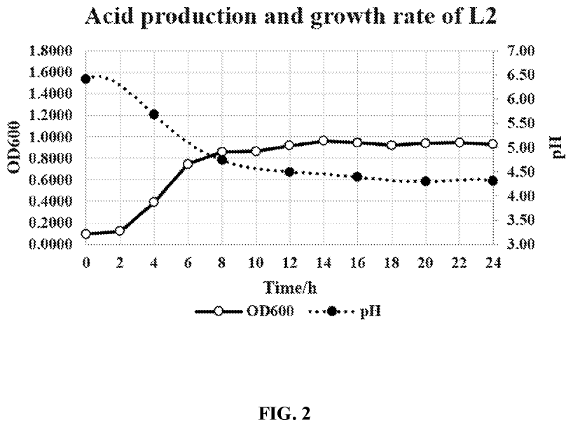

The L2 strain was tested and identified using the same testing method as in the embodiment, and the adopted test method and test material were also the same as those in the embodiment, and the relevant results are shown in Table 2 and FIG. 2.

TABLE 2
The physiological and biochemical characteristics of strain L2

| | |
|---|---|
| Strain | L2 |
| Source | Epiphytic bacteria in alfalfa |
| Shape | Sphere |
| Gram staining | + |
| Catalase | − |
| Fermentation type | Homotype |

Growth temperature and corresponding results

| | |
|---|---|
| 4° C. | w |
| 15° C. | + |
| 30° C. | + |
| 35° C. | + |
| 45° C. | + |

Growth pH value and corresponding results

| | |
|---|---|
| 3.0 | w |
| 3.5 | w |
| 4.0 | w |
| 4.5 | + |
| 9.0 | ++ |

Salt resistance and corresponding results

| | |
|---|---|
| 3% NaCl | + |
| 6.5% NaCl | + |

In the results of Table 2, the “w”, “+”, and “++” in the tolerance test represent the following meanings: w: OD600ₙₘ<0.5; +: 0.5≤OD600ₙₘ<1; ++: 1≤OD600ₙₘ<1.5.

The results in Table 2 and FIG. 2 indicate that the L2 strain is a gram-positive, homofermentative coccus with strong salt tolerance, fast growth rate, and strong comprehensive acid-producing ability.

The 16S rRNA (see SEQ ID NO. 2) gene homology analysis results showed that L2 is more than 99% similar to Lactococcus lactis. Combined with physiological and biochemical indicators, it is determined that L2 is of the same species as Lactococcus lactis.

Comparison of Carbon Source Utilization Rates between Endophytic Pediococcus pentosaceus EL5 and Epiphytic Lactococcus lactis L2 in Alfalfa

Biolog AN MicroPlate (Catalog No. 1007) was used for carbon source utilization rate measurement, and all necessary nutrients and biochemical reagents were pre filled and dried in 96 wells. Tetrazole redox dyes indicate the degree of microbial utilization of carbon sources by microorganisms through changes in chromaticity. Carbon source utilization rate=(48 h absorbance value of corresponding carbon source hole at 590 nm-48 h absorbance value of blank control well at 590 nm)/48 h absorbance value of blank control well at 590 nm.

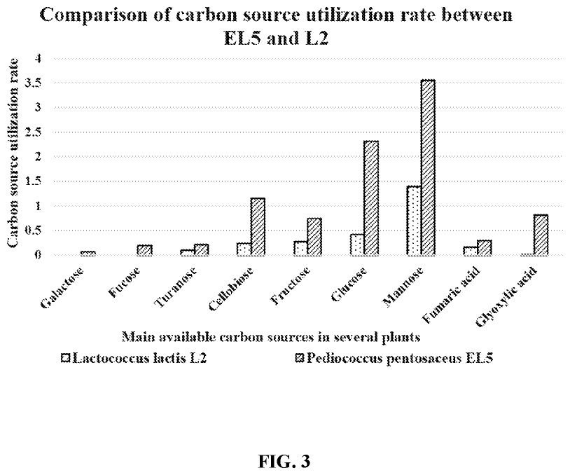

The experimental results showed that the total utilization rates of the main available carbon sources in several plants, such as galactose, fucose, turanose, cellobiose, fructose, dextrose, mannose, fumaric acid, glyoxylic acid, and so on, by endophytic Pediococcus pentosaceus EL5 in alfalfa were higher than that by epiphytic Lactobacillus lactis L2 in alfalfa, see FIG. 3 for details.

### Application Example

Preparation of Silage

The alfalfa harvested by Fengmao Shengyuan Agricultural Technology Co., Ltd. in Huanghua City, Hebei Province, was cut short to 2-3 cm, mixed, and respectively inoculated with the Pediococcus pentosaceus EL5 (EL5 treatment group) of the embodiment and the Lactococcus lactis L2 (L2 treatment group). Add about 1×10⁶ strain EL5 or strain L2 to per gram of fresh material. Load the uniformly mixed alfalfa into 28 cm×35 cm polyethylene silage bags, each treatment is packed in 3 bags, with approximately 500 g each. The bags were vacuumed and sealed with a vacuum sealing machine, stored at a temperature of 20-25° C., and fermented for 7, 30, and 60 days before opening.

Testing of Alfalfa Raw Material and Silage

Samples were taken to analyze the nutritional components of alfalfa raw material and the fermentation quality and nutritional components of alfalfa silage in the EL5 treatment group and the L2 treatment group. The specific results are shown in Tables 3 to 6. The test procedures were as follows:

Composition analysis of silage and raw material samples: Take multiple alfalfa silage samples with each of 20 g, add 180 mL of distilled water and stir them evenly, use a tissue grinder to pulverize them for 1 min, and then filter them with four layers of gauze to filter out the grass residue. The leaching solution obtained was used for the determination of pH value, lactic acid, acetic acid, propionic acid and ammonia nitrogen contents. The pH value of alfalfa silage leaching solution was measured with a PH meter (pHS-3C, Shanghai, China); the contents of lactic acid, acetic acid, and propionic acid were analyzed using high-performance liquid chromatography, with chromatographic column: KC-811 column, detector: SPD-M10AVP, detection wavelength: 210 nm; mobile phase: 3 mmol/L perchloric acid, flow rate: 1 mL/min, injection volume: 5 uL, column temperature 50° C. Ammonia nitrogen was determined using the phenol sodium hypochlorite colorimetric method (Broderick and Kang, 1980). The dry matter contents of alfalfa raw material and silage were determined by drying method. After mixing the samples, they were placed in a blast drying oven at 65° C. for about 48 hours until the quality was constant, and the contents of the dry matter were determined. The dried samples were pulverized by a plant pulverizer. After sieving, it was used for the determination of crude protein, crude fat, neutral detergent fiber, and acidic detergent fiber content; Crude protein was determined by Kjeldahl method, crude fat was determined by petroleum ether extraction method (AOAC, 2010), and neutral and acid detergent fiber contents were determined by Van Soest method (VanSoest et al., 1991).

After the silage was opened at each time point, accurately weigh 20 g of silage sample, put it into a triangular flask containing 180 ml of sterile water, seal it with sealing film, put it into a 180r/min shaking table and shake it for 30 min to fully disperse microorganisms, and let it stand for 10˜40 s to form a 10⁻¹ diluent. Suck 100 μl of the 10⁻¹ dilute, add it to a centrifuge tube containing 900 μl sterile water, and mix thoroughly to form a 10⁻² diluent; then suck 100 μl of 10⁻² diluent, add it to a centrifuge tube containing 900 μl of sterile water, and mix well to obtain 10⁻³ diluent; dilute continuously using the same method to produce a series of diluted bacterial solutions such as 10⁻⁴ and 10⁻⁵. Take 3-compartment culture dishes containing sterile MRS medium, blue light broth agar medium, Bengal red medium, nutrient agar medium (NA), and potato dextrose agar medium (PDA), respectively, and use a marker pen to mark the dilution levels of 10⁻¹, 10⁻³, and 10⁻⁵ on three 120° sectors of each culture dish; then draw 20 μl from the dilution tubes of 10⁻¹, 10⁻³, and 10⁻⁵ respectively, drop in the corresponding sector areas on the surfaces of the culture mediums, and then use coating rods to spread the bacterial solutions evenly on the mediums. The coated culture mediums should stand for 20-30 minutes, put the culture dish with MRS culture medium into an anaerobic box, and put it together with other culture dishes into a 37° C. constant temperature incubator for 48 h, then take it out for counting, and measure the number of lactic acid bacteria, Escherichia coli, yeast, bacteria and true bacteria during the fermentation of silage.

TABLE 3
Chemical and microbial counts of alfalfa before silage

| Item | Alfalfa raw material |
|---|---|
| Lactic acid bacteria (Log₁₀ CFU/g FM) | 6.61 |
| Escherichia coli (Log₁₀ CFU/g FM) | 7.90 |
| Yeast (Log₁₀ CFU/g FM) | 8.28 |
| WSC (g/kg DM) | 23.10 |
| Crude fat (g/kg DM) | 24.64 |
| NDF(g/kg DM) | 394.43 |
| ADF(g/kg DM) | 280.83 |
| Crude protein (g/kg DM) | 244.59 |
| dry matter (g/kg FM) | 356.66 |

FM: fresh matter,
DM: dry matter,
WSC: water soluble carbohydrates,
NDF: neutral detergent fiber,
ADF: acid detergent fiber

TABLE 4
Effects of EL5 and L2 additives on the
fermentation quality of alfalfa silage

| | | | |
|---|---|---|---|
| T | CK | L2 | EL5 |

| | | | | | |
|---|---|---|---|---|---|
| LA | 7 | d | 25.93c | 34.45b | 40.52a |
| (g/kg DM) | 30 | d | 33.93 | 37.01 | 44.21 |
| | 60 | d | 37.39ab | 29.14b | 48.61a |
| AA | 7 | d | 28.86 | 32.87 | 34.09 |
| (g/kg DM) | 30 | d | 28.53 | 25.43 | 23.51 |
| | 60 | d | 29.17 | 38.79 | 24.95 |
| PA | 7 | d | 12.87 | 12.64 | 10.90 |
| (g/kg DM) | 30 | d | 13.64 | 11.98 | 12.32 |
| | 60 | d | 15.06 | 12.94 | 12.72 |
| BA | 7 | d | ND | ND | ND |
| (g/kg DM) | 30 | d | ND | ND | ND |
| | 60 | d | ND | ND | ND |
| pH | 7 | d | 5.69a | 5.37b | 5.19c |
| | 30 | d | 5.44a | 5.05b | 4.87c |
| | 60 | d | 4.91a | 4.93a | 4.72b |
| NH₃—N | 7 | d | 95.76 | 95.74 | 89.07 |
| (g/kg TN) | 30 | d | 148.72a | 134.46ab | 111.27b |
| | 60 | d | 139.62 | 146.73 | 136.83 |

a-c indicates significant differences in different rows (P < 0.05).
ND, below detection level; T: treatment; FM: fresh matter; DM: dry matter; TN: total nitrogen; LA: lactic acid; AA: acetic acid; PA: propionic acid; BA: butyric acid CK: Control; EL5: treatment group with endophytic Pediococcus pentosaceus EL5 in alfalfa additive; L2: treatment group with epiphytic Lactobacillus lactis L2 in alfalfa additive.

TABLE5
Effects of EL5 and L2 additives on
microorganisms in alfalfa silage

| | | | |
|---|---|---|---|
| T | CK | L2 | EL5 |

| | | | | | |
|---|---|---|---|---|---|
| Lactic acid bacteria | 7 | d | 8.03b | 8.58a | 8.88a |
| (Log₁₀ CFU/g FM) | 30 | d | 7.82b | 8.42a | 8.40a |
| | 60 | d | 7.62 | 7.41 | 7.43 |
| Escherichia coli | 7 | d | 6.64a | 5.56b | 5.55b |
| (Log₁₀ CFU/g FM) | 30 | d | 4.53a | ND | ND |
| | 60 | d | ND | ND | ND |
| Yeast | 7 | d | 6.50a | 5.30b | 4.90b |
| (Log₁₀ CFU/g FM) | 30 | d | ND | ND | ND |
| | 60 | d | ND | ND | ND |

a-b indicates significant differences in different rows (P < 0.05); ND, below detection level; T: Treatment; CK: Control; EL5: treatment group with endophytic Pediococcus pentosaceus EL5 in alfalfa additive; L2: treatment group with epiphytic Lactobacillus lactis L2 in alfalfa additive.

TABLE 6
Effects of EL5 and L2 Additives on Nutrient
Composition of Alfalfa silage

| | T | | CK | L2 | EL5 |
|---|---|---|---|---|---|
| Dry matter | 7 | d | 326.80 | 330.55 | 321.46 |
| (g/kg FM) | 30 | d | 329.29 | 331.12 | 335.25 |
| | 60 | d | 342.29a | 322.63b | 327.20b |
| WSC | 7 | d | 8.15a | 7.51ab | 6.17b |
| (g/kg DM) | 30 | d | 6.14 | 6.11 | 6.61 |
| | 60 | d | 7.25 | 6.91 | 7.25 |
| Crude fat | 7 | d | 25.24ab | 22.94b | 27.51a |
| (g/kg DM) | 30 | d | 31.08 | 35.56 | 32.45 |
| | 60 | d | 31.66 | 31.82 | 33.42 |
| NDF | 7 | d | 381.98 | 369.98 | 384.89 |
| (g/kg DM) | 30 | d | 360.09 | 357.49 | 365.15 |
| | 60 | d | 352.65 | 368.80 | 364.74 |
| ADF | 7 | d | 280.25 | 271.59 | 280.54 |
| (g/kg DM) | 30 | d | 263.02 | 266.24 | 274.98 |
| | 60 | d | 264.07 | 274.04 | 269.27 |
| Crude protein | 7 | d | 238.96b | 241.81ab | 246.80a |
| (g/kg DM) | 30 | d | 235.65 | 237.59 | 239.22 |
| | 60 | d | 234.41 | 234.68 | 240.12 |

a-b indicates significant differences in different rows (P < 0.05), T: Treatment; FM: fresh matter; DM: dry matter.
CK: Control; EL5: treatment group with endophytic Pediococcus pentosaceus EL5 in alfalfa additive; L2: treatment group with epiphytic Lactobacillus lactis L2 in alfalfa additive.

From the results in Table 3-6, it can be seen that compared with the treatment of epiphytic Lactobacillus lactis L2 in alfalfa, endophytic Pediococcus pentosaceus EL5 in alfalfa as an additive can rapidly activate and increase the number of lactic acid bacteria in the early stage of silage fermentation, thus rapidly increasing the content of lactic acid and reducing its pH value, while inhibiting the production of large amounts of ammonia nitrogen and the reproduction of Escherichia coli, yeast and mold. In addition, at 60 days, the protein retention rate of adding EL5 reached 98.17%, which increased the storage capacity per kilogram of protein by 22.22 grams compared to L2 and 23.32 grams compared to CK. Therefore, as a new additive for rapid start-up of alfalfa silage fermentation, Pediococcus pentosaceus EL5 can significantly improve the quality of alfalfa silage.

The above is only the preferred specific implementation of the present application, but the scope of protection of the present application is not limited to this. Any changes or replacements that can be easily thought of by technical personnel familiar with the technical field within the scope of disclosure of the present application should be covered within the scope of protection of the present application.

What is claimed is:

1. A Pediococcus pentosaceus EL5 with the deposit number of CGMCC No. 23923.

2. The Pediococcus pentosaceus EL5 according to claim 1, comprising 16S rRNA as shown in SEQ ID NO. 1.

3. The Pediococcus pentosaceus EL5 according to claim 1, it is isolated from the interior of alfalfa.

4. A silage, comprising a Pediococcus pentosaceus EL5;
wherein the Pediococcus pentosaceus EL5 comprises 16S rRNA as shown in SEQ ID NO. 1.

5. The silage according to claim 4, it is alfalfa silage.

6. A preparation method of a silage, comprising: mixing silage raw material with a Pediococcus pentosaceus EL5 and fermenting to produce the silage;
wherein the silage comprises the Pediococcus pentosaceus EL5, and the Pediococcus pentosaceus EL5 comprises 16S rRNA as shown in SEQ ID NO. 1.

7. The method according to claim 6, wherein the ratio of the number of viable strains of Pediococcus pentosaceus EL5 to the mass of the silage raw material is 1.0×10⁵˜2.0×10⁶ CFU/g.

8. The method according to claim 6, wherein the silage raw material comprises alfalfa.
